(19) United States
(12) Patent Application Publication
(10) Pub. No.: US 2019/0241445 A1
(43) Pub. Date: Aug. 8, 2019

(54) ULTRAVIOLET IRRADIATION DEVICE

(71) Applicant: NIKKISO CO., LTD., Tokyo (JP)

(72) Inventor: Shinya WATANABE, Hakusan-shi (JP)

(21) Appl. No.: 16/389,651

(22) Filed: Apr. 19, 2019

Related U.S. Application Data
(63) Continuation of application No. PCT/JP2017/037202, filed on Oct. 13, 2017.

(30) Foreign Application Priority Data
Oct. 19, 2016 (JP) 2016-205528

(51) Int. Cl. C02F 1/32; A61L 2/26; A61L 2/10

(52) U.S. Cl. CPC C02F 1/325; A61L 2/26; A61L 2/10; A61L 2202/11; C02F 2201/3227; C02F 2201/3228; A61L 2202/122; C02F 2201/3222

(57) ABSTRACT

An ultraviolet irradiation device includes: a pipe joint including a first connection part connectable to a first pipe extending in a first direction, a second connection part connectable to a second pipe extending in a second direction intersecting the first direction, and a main part positioned between the first connection part and the second connection part; and a light source configured to radiate ultraviolet light from the main part toward the interior of the first pipe. The ultraviolet irradiation device may include a window member provided in the main part and transmitting the ultraviolet light from the light source.

### RELATED APPLICATION

Priority is claimed to Japanese Patent Application No. 2016-205528, filed on Oct. 19, 2016, the entire content of which is incorporated herein by reference.

### BACKGROUND OF THE INVENTION

### 1. Field of the Invention

The present invention relates to an ultraviolet irradiation device.

### 2. Description of the Related Art

It is known that ultraviolet light has sterilization capability. Devices that radiate ultraviolet light are used for sterilization in medical and food processing fronts. Devices that sterilize a fluid such as water continuously by irradiating the fluid with ultraviolet light are also used. One example is a device in which an ultraviolet LED is provided on the inner wall of a flow passage formed by a metal pipe.

In equipment for supplying a liquid such as potable water, the liquid is supplied via a discharge port. Generally, the discharge port is exposed outside. Therefore, bacteria or organic substance infiltrating the equipment from outside may result in proliferation of miscellaneous bacteria on the inner wall of the pipe of the discharge port. If the liquid is supplied in the presence of miscellaneous bacteria that have proliferated, the liquid containing miscellaneous bacteria will be supplied.

### SUMMARY OF THE INVENTION

In this background, one illustrative purpose of the present invention is to provide a technology of sterilizing the interior of a pipe such as a discharge port properly.

An ultraviolet irradiation device according to an embodiment includes: a pipe joint including a first connection part connectable to a first pipe extending in a first direction, a second connection part connectable to a second pipe extending in a second direction intersecting the first direction, and a main part positioned between the first connection part and the second connection part; and a light source configured to radiate ultraviolet light from the main part toward the interior of the first pipe.

According to this embodiment, the interior of the first pipe can be irradiated with ultraviolet light and sterilized, by using the ultraviolet irradiation device according to the embodiment in a portion of connection between the first pipe and the second pipe. In the case the first pipe is a discharge port, for example, the discharge port can be irradiated with ultraviolet light and sterilized. Also, configuring the irradiation device as a pipe joint type makes it easy to rejoin pipes or replace a pipe or a pipe joint that has undergone aged deterioration. Accordingly, the convenience is increased.

The ultraviolet irradiation device may further include a window member provided in the main part and transmitting the ultraviolet light from the light source.

The light source may include a light emitting device that outputs ultraviolet light and an optical mechanism that regulates a direction of radiation of the ultraviolet light output from the light emitting device.

The optical mechanism may include a reflecting member provided to surround the light emitting device.

The main part may define a connection flow passage bent from the first connection part to the second connection part, and the light source may be configured to radiate the ultraviolet light toward an inner corner of the connection passage bent.

The first connection part may be configured to be connectable to the first pipe such that a leading end of the first pipe does not overlap the inner corner of the connection flow passage, and the second connection part may be configured to be connectable to the second pipe such that a leading end of the second pipe does not overlap the inner corner of the connection flow passage.

The light source may be provided at a position shifted from a central axis of the first pipe toward the inner corner.

The light source may be positioned on a central axis of the first pipe.

The ultraviolet irradiation device may further include: the first pipe connected to the first connection part, and the first pipe may be made of a fluororesin material and include a discharge port for a fluid flowing in the main part.

The pipe joint may be an L-shaped joint, the light source may be a first light source, and the ultraviolet irradiation device may further include: a second light source configured to radiate ultraviolet light from the main part toward the interior of the second pipe.

The pipe joint may be a branch connection, and the ultraviolet irradiation device may further include: a third connection part connected to a third pipe extending in a third direction intersecting the first direction.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 schematically shows a configuration of an ultraviolet irradiation device according to an embodiment;

FIG. 2 is a cross-sectional view schematically showing a configuration of the ultraviolet irradiation device according to variation 1;

FIG. 3 is a cross-sectional view schematically showing a configuration of a ultraviolet irradiation device according to variation 2;

FIG. 4 is a cross-sectional view schematically showing a configuration of an ultraviolet irradiation device according to variation 3;

FIG. 5 is a cross-sectional view schematically showing a configuration of an ultraviolet irradiation device according to variation 4; and

FIG. 6 is a cross-sectional view schematically showing a configuration of an ultraviolet irradiation system according to the embodiment.

### DETAILED DESCRIPTION OF THE INVENTION

The invention will now be described by reference to the preferred embodiments. This does not intend to limit the scope of the present invention, but to exemplify the invention.

A detailed description will be given of embodiments of the present invention with reference to the drawings. Like numerals are used in the description to denote like elements and a duplicate description is omitted as appropriate.

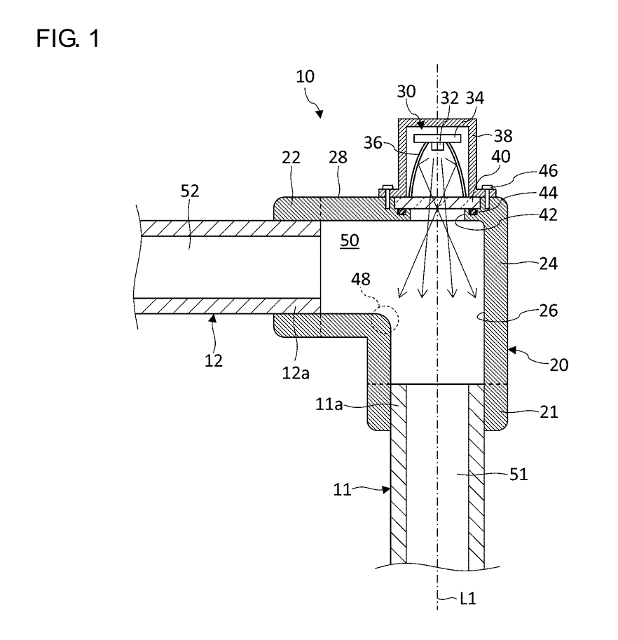

FIG. 1 schematically shows a configuration of an ultraviolet irradiation device 10 according to an embodiment. The ultraviolet irradiation device 10 includes a pipe joint 20, a light source 30, and a window member 40. The ultraviolet irradiation device 10 is a device of a pipe joint type that connects a first pipe 11 and a second pipe 12. The ultraviolet irradiation device 10 irradiates the interior of the first pipe 11 connected to the ultraviolet irradiation device 10 so as to sterilize the inner wall of the first pipe 11.

The pipe joint 20 includes a first connection part 21, a second connection part 22, and a main part 24. The first connection part 21 is a part connected to the first pipe 11 extending in the first direction, and the second connection part 22 is a part connected to the second pipe 12 extending in the second direction intersecting the first direction. The main part 24 is a part positioned between the first connection part 21 and the second connection part 22 and defining a connection flow passage 50 bent from the first connection part 21 to the second connection part 22. In the illustrated example, the pipe joint 20 is configured as an L-shaped joint angled at 90°, but the direction of connection of the pipe joint 20 need not necessarily be 90°. In one variation, the first direction and the second direction may intersect at an acute angle smaller than 90° or a blunt angle larger than 90°.

The pipe joint 20 is made of a metal material or a resin material. It is preferred that the pipe joint 20 be made of a material having a high durability and shielding capability for the ultraviolet light output from the light source 30. Such a material is exemplified by a metal material such as stainless steel and aluminum and a fluororesin material such as polytetrafluoroethylene (PTFE). A material having a high ultraviolet reflectivity may be used in a part of an inner surface 26 of the main part 24 of the pipe joint 20 directly irradiated with the ultraviolet light from the light source 30.

The first connection part 21 receives a first leading end 11a of the first pipe 11 so as to connect the connection flow passage 50 in the main part 24 with a first flow passage 51 in the first pipe 11 in a watertight manner. The second connection part 22 receives a second leading end 12a of the second pipe 12 so as to connect the connection flow passage 50 with a second flow passage 52 in the second pipe 12 in a watertight manner. The first connection part 21 and the second connection part 22 may have a connection structure for connection with the first leading end 11a and the second leading end 12a, respectively. For example, the first connection part 21 and the second connection part 22 may have a threaded structure, a flange structure, or a one-touch connection structure.

The first connection part 21 is configured to be connected to the first pipe 11 such that the first leading end 11a does not overlap an inner corner 48 of the bent connection flow passage 50. Similarly, the second connection part 22 is configured to be connected to the second pipe 12 such that the second leading end 12a does not overlap the inner corner 48 of the bent connection flow passage 50. By causing the leading ends of the connected pipes not to overlap the inner corner 48, the liquid is prevented from staying between the leading end of the pipe and the inner corner 48 to promote proliferation of bacteria near the inner corner 48.

The light source 30 is fitted to the main part 24 and is provided to radiate ultraviolet light from the main part 24 toward the interior of the first pipe 11. The light source 30 is positioned on the central axis L1 of the first pipe 11 to radiate ultraviolet light in the first direction aligned with the central axis L1. The light source 30 is provided outside the main part 24 and radiates ultraviolet light toward the interior of the main part 24 via the window member 40 provided in the main part 24.

The light source 30 includes a light emitting device 32, a substrate 34, and a reflector 36. The light emitting devices 32 is a so-called ultra violet-light emitting diode (UV-LED) and outputs deep ultraviolet light having a central wavelength or a peak wavelength included in a range of about 200 nm˜350 nm. It is preferred that the light emitting device 32 emit ultraviolet light near 260 nm˜290 nm having a high sterilizing efficiency. Such an ultraviolet LED is exemplified by an aluminum gallium nitride (AlGaN) based LED. The light emitting device 32 is mounted on the substrate 34. It is preferred that the substrate 34 be made of a highly exoergic material. For example, the substrate 34 is made of a base material such as copper (Cu) and aluminum (Al).

The reflector 36 is a reflecting member of a bowl shape provided to surround the light emitting device 32, and the light emitting device 32 is provided at the bottom of the bowl shape. The reflector 36 is an optical mechanism for regulating the direction of radiation of ultraviolet light from the light emitting device 32 and causes the direction of radiation of ultraviolet light output from the light emitting device 32 to be aligned with the central axis L1 of the first pipe 11. This allows the deep ultraviolet light output from the light emitting device 32 to be incident on the interior of the first pipe 11 efficiently and increases the sterilization efficiency. The reflector 36 causes a portion of the ultraviolet light from the light emitting device 32 to be radiated toward the inner corner 48 of the connection flow passage 50. In this way, the inner corner 48, where the flow of the fluid is likely to stay in the main part 24 to promote proliferation of bacteria, is suitably sterilized. In one variation, a refracting optical device such as a lens may be used in place of the reflector 36, which is a reflecting optical device, to regulate the direction of radiation from the light emitting device 32.

A cover 38 is provided around the light source 30. The cover 38 protects the light source 30 and prevents the ultraviolet light from the light source 30 from leaking outside. The cover 38 is fitted to an outer surface 28 of the main part 24 by means of a fixing member 46 such as a screw. Like the pipe joint 20, the cover 38 is made of a metal material or a resin material and is preferably made of a material having a high durability for ultraviolet light and a high shielding capability for ultraviolet light. A power supply (not shown) for lighting the light source 30 may be provided in the cover 38. The light source 30 may receive the supply of electric power via a power supply cable (not shown) extending outside the cover 38.

The window member 40 is provided between the connection flow passage 50 and the light source 30. The window member 40 is made of a material having a high ultraviolet transmittance such as quartz (SiO₂), sapphire (Al₂O₃), and amorphous fluororesin. The main part 24 is provided with an opening 42 on the central axis L1 of the first pipe 11, and the window member 40 is fitted to the main part 24 to block the opening 42. A seal member 44 is provided between the window member 40 and the main part 24. In the illustrated example, the window member 40 is sandwiched and fixed between the main part 24 and the cover 38. A step produced by the thickness of the opening 42 is provided between the inner surface 26 of the main part 24 and the window member 40, and the window member 40 is provided such that the position of the window member 40 recedes when viewed from the connection flow passage 50.

According to the configuration described above, the interior of the first pipe 11 is irradiated with ultraviolet light to sterilize the inner wall of the first pipe 11, by using the ultraviolet irradiation device 10 to connect the first pipe 11 and the second pipe 12. Also, the fluid flowing in the connection flow passage 50 in the pipe joint 20 is irradiated with ultraviolet light to sterilize the fluid. Further, proliferation of bacteria in the pipe joint 20 is inhibited by radiating ultraviolet light toward the inner corner 48 in which the flow of the fluid is slow. Since the recess (the opening 42) of the window member 40 in which the fluid is likely to stay is also irradiated with high-intensity ultraviolet light, proliferation of bacteria in the pipe joint 20 is inhibited. Thus, the connection flow passage 50 and the first flow passage 51 are maintained in a sanitized condition according to the embodiment.

In accordance with the embodiment, the ultraviolet irradiation device 10, the first pipe 11, and the second pipe 12 can be easily disassembled. In the event that any of the members is degraded, only the relevant parts can be replaced easily for maintenance. Since it is easy to disassembly and clean the device periodically, the components forming the flow passage can be used in a sanitized condition over a long period of time.

In the embodiment, the pipe joint 20 and the window member 40 are described as being formed as separate parts. In one variation, the window member 40 and the pipe joint 20 may be integrated.

In the embodiment, the light source 30 is described as being provided outside the pipe joint 20. In one variation, the light source 30 may be provided in the pipe joint 20, or the light source 30 may be embedded in the wall surface of the pipe joint 20.

### Variation 1

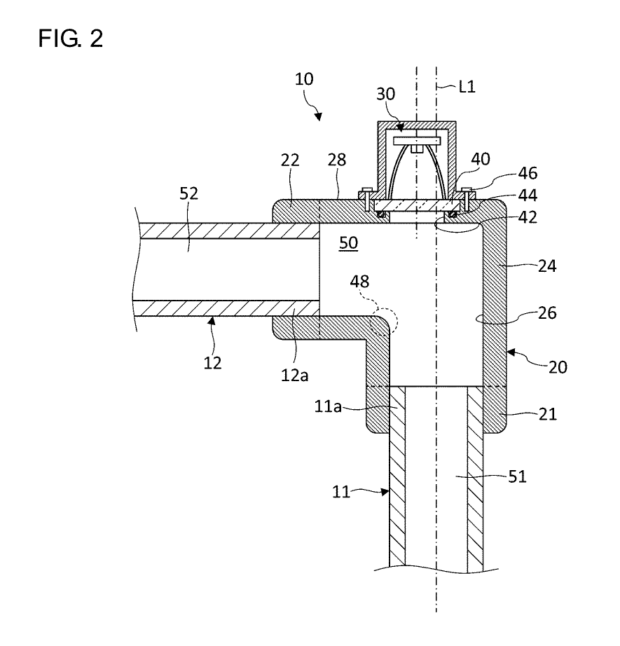

FIG. 2 is a cross-sectional view schematically showing a configuration of the ultraviolet irradiation device 10 according to variation 1. This variation differs from the embodiment described above in that the light source 30 is provided at a position shifted from the central axis L1 of the first pipe 11. The light source 30 is provided at a position shifted from the central axis L1 of the first pipe 11 toward the inner corner 48 and is positioned closer to the second connection part 22 than in the embodiment described above. Similarly, the position where the opening 42 is provided is also shifted from the central axis L1 of the first pipe 11. According to this variation, the quantity of ultraviolet light traveling from the light source 30 toward the inner corner 48 is increased so that the sterilization effect in the inner corner 48 is further increased.

### Variation 2

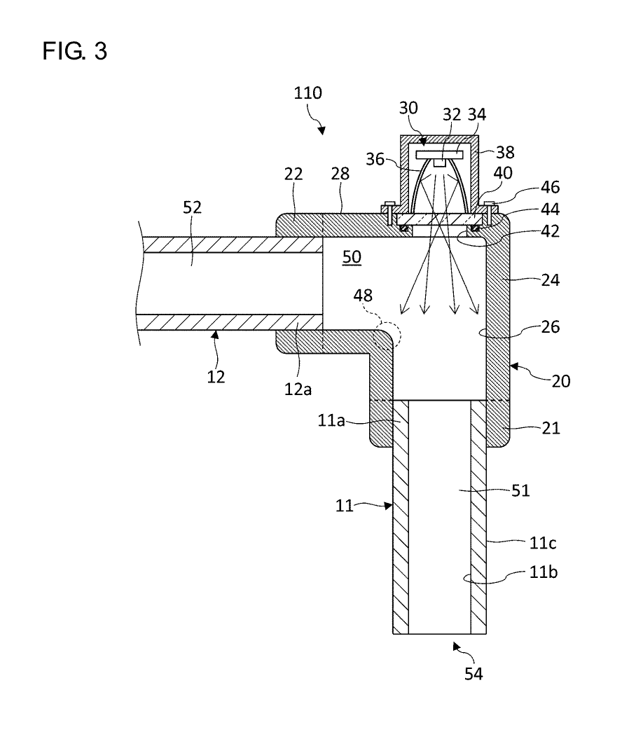

FIG. 3 is a cross-sectional view schematically showing a configuration of a ultraviolet irradiation device 110 according to variation 2. The ultraviolet irradiation device 110 according to the variation includes the first pipe 11, the pipe joint 20, the light source 30, and the window member 40. In other words, the ultraviolet irradiation device 110 includes the first pipe 11 in addition to the features of the ultraviolet irradiation device 10 shown in FIG. 1. The first pipe 11 includes a discharge port 54 for supplying the fluid outside. The first pipe 11 may be separated from the pipe joint 20 or may be integrated with the pipe joint 20.

The first pipe 11 is made of a fluororesin material such as PTFE. The first pipe 11 made of PTFE allows a high-intensity ultraviolet light to be radiated in the axial direction of the first pipe 11 by reflecting the ultraviolet light on an inner wall surface 11b of the first pipe 11. By causing the PTFE pipe wall to have a thickness of 5 mm or larger, for example, the ultraviolet reflectivity on the inner wall surface 11b is increased. Meanwhile, a portion of the ultraviolet light may be allowed to leak from an PTFE outer wall surface 11c by causing the PTFE pipe wall to have a thickness of smaller than 5 mm, the For example, ultraviolet light may be allowed to leak to the outer wall surface 11c of the first pipe 11 by causing the PTFE pipe wall to have a thickness of about 1 mm˜2 mm so that the outer wall surface 11c is sterilized with ultraviolet light.

According to the variation, at least one of the inner wall surface 11b and the outer wall surface 11c of the first pipe 11 forming the discharge port 54 is suitably sterilized. The discharge port 54 is exposed outside and so may provide a basis for bacterial growth in the event of invasion of bacteria or organic matter from outside. Bacteria proliferating in the discharge port 54 may spread far into the flow passage via the first pipe 11. According to the variation, the discharge port 54, that may provide a basis for bacterial proliferation, is suitably sterilized so that not only the discharge port 54 is sterilized but also upstream spread of bacteria in the flow passage is inhibited. Thus, according to the variation, the entirety of the flow passage leading to the discharge port 54 is maintained in a sanitized condition.

### Variation 3

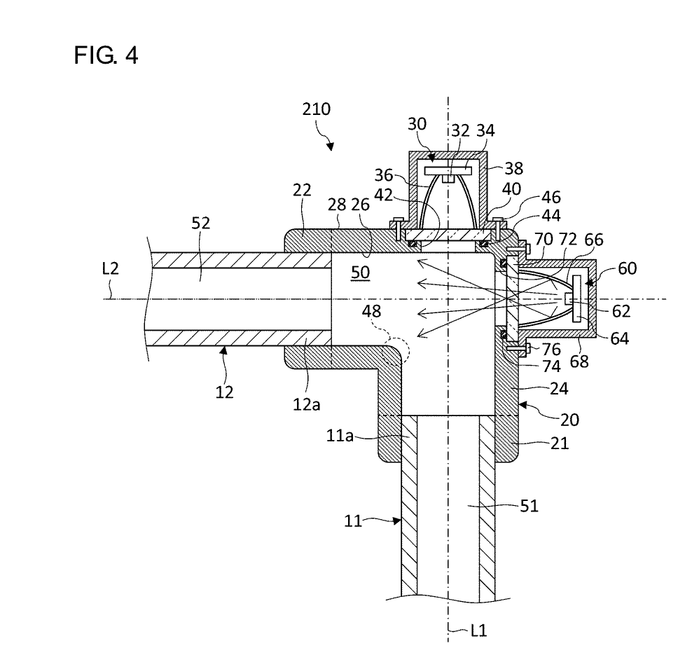

FIG. 4 is a cross-sectional view schematically showing a configuration of an ultraviolet irradiation device 210 according to variation 3. The ultraviolet irradiation device 210 includes a pipe joint 20, a light source 30 (also referred to as the first light source 30), a window member 40 (also referred to as the first window member 40), a second light source 60, and a second window member 70. This variation differs from the embodiment described above in that the second light source 60 for irradiating the interior of the second pipe 12 with ultraviolet light is provided in addition to the first light source 30 for irradiating the interior of the first pipe 11 with ultraviolet light.

The second light source 60 is provided in the main part 24 and is provided to radiate ultraviolet light from the main part 24 toward the interior of the second pipe 12. The second light source 60 is positioned on the central axis L2 of the second pipe 12 to radiate ultraviolet light in the second direction along the central axis L2. Like the first light source 30, the second light source 60 includes a light emitting device 62, a substrate 64, and a reflector 66. A second cover 68 is provided around the second light source 60. The second cover 68 is fitted to an outer surface 28 of the main part 24 by means of a fixing member 76.

The second window member 70 is provided between the connection flow passage 50 and the second light source 60. The second window member 70 is fitted so as to block a second opening 72 provided on the central axis L2 of the second pipe 12. A seal member 74 is sandwiched between the main part 24 and the second window member 70 in a water tight manner.

According to this variation, not only the first light source 30 irradiates the interior of the first pipe 11 with ultraviolet light but also the second light source 60 irradiates the interior of the second pipe 12 with ultraviolet light. This further increases the sterilization effect in the pipes connected to the ultraviolet irradiation device 210.

In a further variation, the second light source 60 may be provided at a position shifted from the central axis L2 of the second pipe 12 toward the inner corner 48. In this way, the inner corner 48 of the bent connection flow passage 50 may be irradiated with high-intensity ultraviolet light from the second light source 60.

### Variation 4

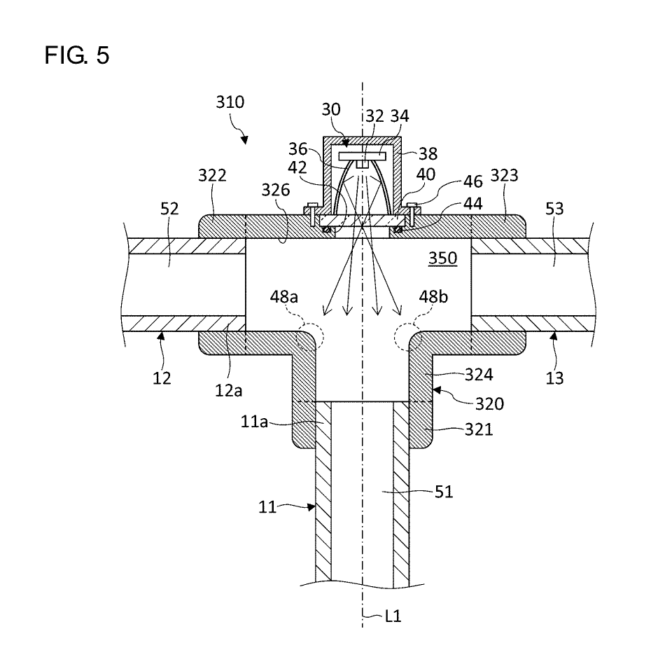

FIG. 5 is a cross-sectional view schematically showing a configuration of an ultraviolet irradiation device 310 according to variation 4. This variation differs from the embodiment described above in that a pipe joint 320 is a branch connection configured to connect three pipes 11, 12, and 13. The following description highlights the difference from the embodiment described above.

The ultraviolet irradiation device 310 includes a pipe joint 320, a light source 30, and a window member 40. The pipe joint 320 includes a first connection part 321, a second connection part 322, a third connection part 323, and a main part 324. The first connection part 321 is a part connected to the first pipe 11 extending in the first direction, and the second connection part 322 is a part connected to the second pipe 12 extending in the second direction intersecting the first direction, and the third connection part 323 is a part connected to the third pipe 13 extending in the third direction intersecting the first direction. The main part 324 is positioned between the first connection part 321, the second connection part 322, and the third connection part 323.

In the illustrated example, the pipe joint 320 has a shape of a letter T and is configured such that the central axes of the second pipe 12 and the third pipe 13 are aligned. In other words, the second direction and the third direction are parallel. In a further variation, the second direction and the third direction may not be parallel. For example, the pipe joint 320 may have a shape of a letter Y. In a still further variation, the pipe joint 320 may have a shape such that the first direction is the x direction, the second direction is the y direction, and the third direction is the z direction.

The main part 324 defines a connection flow passage 350 that connects the first flow passage 51 in the first pipe 11, the second flow passage 52 in the second pipe 12, and the third flow passage 53 in the third pipe 13. The connection flow passage 350 is provided with a first inner corner 48a which is an inner corner of the flow passage bent from the first connection part 321 toward the second connection part 322 and with a second inner corner 48b which is an inner corner of the flow passage bent from the first connection part 321 toward the third connection part 323.

The light source 30 is provided on the central axis L1 of the first pipe 11 and is arranged to radiate ultraviolet light in the first direction toward the interior of the first pipe 11. The light source 30 is arranged to irradiate at least a portion of an inner surface 326 of the main part 324, and, in particular, the first inner corner 48a and the second inner corner 48b. The window member 40 is provided between the light source 30 and the connection flow passage 350 and is positioned on the central axis L1 of the first pipe 11. The window member 40 transmits the ultraviolet light radiated from the light source 30 toward the interior of the first pipe 11.

According to the configuration described above, the interior of one of the three pipes connected to the branch connection is irradiated with ultraviolet light for sterilization. In a further variation, the pipe joint 320 may be configured such that four or more pipes can be connected. Alternatively, a plurality of light sources may be provided so that the interior of two or more of the three or more pipes connected to the branch connection can be irradiated with ultraviolet light.

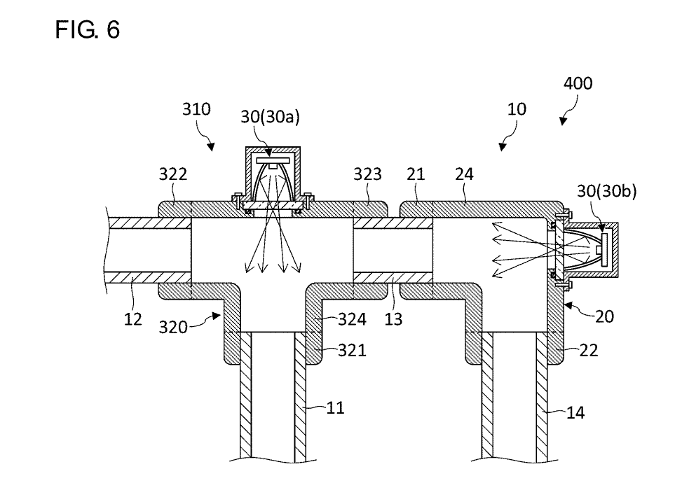

FIG. 6 is a cross-sectional view schematically showing a configuration of an ultraviolet irradiation system 400 according to the embodiment. The ultraviolet irradiation system 400 is built by combining a plurality of the ultraviolet irradiation devices 10, 110, 210, and 310 according to the embodiment or the variations. For example, the ultraviolet irradiation system 400 may be provided with the ultraviolet irradiation device 310 shown in FIG. 5 (also referred to as the first ultraviolet irradiation device 310) and the ultraviolet irradiation device 10 shown in FIG. 1 (also referred to as the second ultraviolet irradiation device 10).

The first pipe 11, the second pipe 12, and the third pipe 13 are connected to the first ultraviolet irradiation device 310. In the first ultraviolet irradiation device 310, the first pipe 11 is connected to the first connection part 321, the second pipe 12 is connected to the second connection part 322, and the third pipe 13 is connected to the third connection part 323. The third pipe 13 and a fourth pipe 14 are connected to the second ultraviolet irradiation device 10. In the second ultraviolet irradiation device 10, the third pipe 13 is connected to the first connection part 21, and the fourth pipe 14 is connected to the second connection part 22.

The light source 30 provided in the first ultraviolet irradiation device 310 (also referred to as a first light source 30a) irradiates the interior of the first pipe 11 with ultraviolet light. The light source 30 provided in the second ultraviolet irradiation device 10 (also referred to as a second light source 30b) irradiates the interior of the third pipe 13 with ultraviolet light. The ultraviolet light radiated by the second light source 30b enters the interior of the main part 324 of the first ultraviolet irradiation device 310 via the third pipe 13 and irradiates the interior of the second pipe 12. Accordingly, the embodiment ensures that the pipe that cannot be irradiated by the ultraviolet irradiation device 310 alone is irradiated by a further ultraviolet irradiation device 10 with ultraviolet light, thereby expanding the range subject to sterilization.

Described above is an explanation based on an exemplary embodiment. The embodiment is intended to be illustrative only and it will be understood by those skilled in the art that various design changes are possible and various modifications are possible and that such modifications are also within the scope of the present invention.

It should be understood that the invention is not limited to the above-described embodiment, but may be modified into various forms on the basis of the spirit of the invention. Additionally, the modifications are included in the scope of the invention.

What is claimed is:

1. An ultraviolet irradiation device comprising:
a pipe joint including a first connection part connectable to a first pipe extending in a first direction, a second connection part connectable to a second pipe extending in a second direction intersecting the first direction, and a main part positioned between the first connection part and the second connection part; and
a light source configured to radiate ultraviolet light from the main part toward the interior of the first pipe.

2. The ultraviolet irradiation device according to claim 1, further comprising:
a window member provided in the main part and transmitting the ultraviolet light from the light source.

3. The ultraviolet irradiation device according to claim 1, wherein
the light source includes a light emitting device that outputs ultraviolet light and an optical mechanism that regulates a direction of radiation of the ultraviolet light output from the light emitting device.

4. The ultraviolet irradiation device according to claim 3, wherein
the optical mechanism includes a reflecting member provided to surround the light emitting device.

5. The ultraviolet irradiation device according to claim 1, wherein
the main part defines a connection flow passage bent from the first connection part to the second connection part, and
the light source is configured to radiate the ultraviolet light toward an inner corner of the connection passage bent.

6. The ultraviolet irradiation device according to claim 5, wherein
the first connection part is configured to be connectable to the first pipe such that a leading end of the first pipe does not overlap the inner corner of the connection flow passage, and
the second connection part is configured to be connectable to the second pipe such that a leading end of the second pipe does not overlap the inner corner of the connection flow passage.

7. The ultraviolet irradiation device according to claim 5, wherein
the light source is provided at a position shifted from a central axis of the first pipe toward the inner corner.

8. The ultraviolet irradiation device according to claim 1, wherein
the light source is positioned on a central axis of the first pipe.

9. The ultraviolet irradiation device according to claim 1, further comprising:
the first pipe connected to the first connection part, wherein
the first pipe is made of a fluororesin material and includes a discharge port for a fluid flowing in the main part.

10. The ultraviolet irradiation device according to claim 1, wherein
the light source is a first light source,
the ultraviolet irradiation device further comprising:
a second light source configured to radiate ultraviolet light from the main part toward the interior of the second pipe.

11. The ultraviolet irradiation device according to claim 10, wherein
the second light source is provided on a central axis of the second pipe and is configured to radiate ultraviolet light in the second direction.

12. The ultraviolet irradiation device according to claim 10, wherein
the second light source is provided at a position shifted from a central axis of the second pipe toward an inner corner and is configured to radiate the ultraviolet light toward the inner corner in the second direction.

13. The ultraviolet irradiation device according to claim 10, wherein
the pipe joint is an L-shaped joint.

14. The ultraviolet irradiation device according to claim 1, wherein
the pipe joint is a branch connection,
the ultraviolet irradiation device further including:
a third connection part connected to a third pipe extending in a third direction intersecting the first direction.

15. The ultraviolet irradiation device according to claim 1, wherein
the main part defines a connection flow passage bent from the first connection part to the second connection part,
the light source is provided at a position shifted from a central axis of the first pipe toward an inner corner of the connection passage bent and is configured to radiate the ultraviolet light toward the inner corner in the first direction.

16. The ultraviolet irradiation device according to claim 15, wherein
the light source includes a single light emitting device, and the single light emitting device is provided at a position shifted from the central axis of the first pipe toward the inner corner.

17. The ultraviolet irradiation device according to claim 16, wherein
the light source further includes a reflector provided to surround the single light emitting device and reflecting the ultraviolet light from the single light emitting device in the first direction.
